(19) United States
(12) Patent Application Publication
(10) Pub. No.: US 2009/0312683 A1
(43) Pub. Date: Dec. 17, 2009

(54) Knee rehabilitation exercise device

(76) Inventor: Jebodiah Burns, Santa Clara, CA (US)

Correspondence Address: JEBODIAH BURNS, 670 BUCHER AVE, SANTA CLARA, CA, 95051, US

(21) Appl. No.: 12/157,767

(22) Filed: Jun. 13, 2008

(51) Int. Cl. A61F 5/00; A63B 21/02

(52) U.S. Cl. 602/26; 428/124

(57) ABSTRACT

A knee rehabilitation exercise device comprised of a right and left leg brace, a rear knee strap, an upper leg strap assembly, a lower leg strap assembly, a portable rear brace assembly and an elastic resistance band. The left and right leg brace members are each comprised of an upper member and a lower member rotatably pinned together at the area of the knee joint. The left and right leg braces are attached to the upper and lower leg strap assembly. The rear knee strap is attached to the left and right leg braces at the knee joint. When a user places the elastic band onto L hooks mounted on the upper and lower strap assembly and the rear brace assembly is placed between the rear of the user's knee and the elastic band, a resistive stretching is caused when the user straightens his leg which exercises and strengthens the user's leg muscles in the area of the knee joint.

### CROSS REFERENCE TO RELATED APPLICATIONS

Not Applicable

### STATEMENT REGARDING FEDERALLY SPONSORED RESEARCH OR DEVELOPMENT

Not Applicable

### DESCRIPTION OF ATTACHED APPENDIX

Not Applicable

### BACKGROUND OF THE INVENTION

This invention relates generally to the field of portable leg exercise devices and more specifically to a knee rehabilitation exercise device.

With today's rigorous sports activities including football, tennis, basketball and soccer it is unfortunately a common occurrence for active people to injure their knee joint to the point where surgical treatment is necessary to repair the knee and areas around the knee.

Because of this, it has been necessary to provide devices to help rehabilitate the knee area after surgery. Some devices are simply leg braces that help take-pressure off the knee joint while it is healing. Other devices are machines or apparatus that help strengthen the leg muscles around the knee and associated with proper knee function.

Patents that describe leg braces that help keep the knee joint supported during exercise include U.S. Pat. Nos. 4,982,732, 5,116,296, 4,433,679 and 5,980,435. Patents that describe exercise apparatus for helping to rehabilitate the muscles of the leg around the knee joint include U.S. Pat. Nos. 4,546,968, 4,776,587, 4,979,737 and 5,181,895. However, these patents and other commercially available exercise devices have a deficiency in that none of them allows a person to wear a light weight inexpensive brace which has hooks attached to allow the user to releasably attach an elastic band from the area of the upper leg to the area of the lower leg, and to insert a portable assembly behind the user's knee for the elastic band to stretch over to increase the resistance factor to the muscles surrounding the knee joint. Furthermore, none of the patents or other prior art shows a leg brace exercise device that allows the user to easily and quickly change the resistance band location from the back of the leg to the front of the leg whereby the user can insert a portable assembly in front of the knee which the elastic band passes over to passively stretch the knee joint to a straight position.

### BRIEF SUMMARY OF THE INVENTION

The primary object of the invention is to provide a knee rehabilitation exercise device that allows a person to strengthen leg muscles after knee surgery.

Another object of the invention is to provide a knee rehabilitation exercise device that helps increase the range of knee joint motion.

Another object of the invention is to provide a knee rehabilitation exercise device that can be easily used by a person without the need for additional help from a caregiver.

A further object of the invention is to provide a knee rehabilitation exercise device that is lightweight and can be adjusted to fit a variety of leg lengths and diameters.

Yet, another object of the invention is to provide a knee rehabilitation exercise device that can be fastened to a user's leg quickly and easily.

Other objects and advantages of the present invention will become apparent from the following descriptions, taken in connection with the accompanying drawings, wherein, by way of illustration and example, an embodiment of the present invention is disclosed.

In accordance with a preferred embodiment of the invention, there is disclosed a knee rehabilitation exercise device comprising: a left rigid leg brace, a right rigid leg brace, a rear knee strap, an upper leg strap assembly, a lower leg strap assembly, a portable rear brace assembly, an elastic resistance band, said left and right leg brace members each comprised of an upper member and a lower member rotatably pinned together at the area of the knee joint, said left and right leg brace attached at their uppermost ends to said upper leg strap assembly, said left and right leg brace attached at their lowermost ends to said lower leg strap assembly, said upper leg strap assembly including a rear facing upwardly turned L hook, said lower leg strap assembly including a rear facing downwardly turned L hook, said upper and lower leg strap assemblies able to be formed into a circular shape by a user so that said upper leg strap assembly fits around said user's thigh and said lower leg strap assembly fits around the user's ankle, said upper and lower leg strap assemblies secured to said user's leg by standard attachment means, said rear knee strap fixedly held at said pinned knee joint by said left and right leg brace, said rear brace assembly held in place behind said user's knee joint by said elastic resistance band when said upper and lower L hooks interact with an upper and a lower aperture in said band, so that when said user straightens his knee from a bent position, said elastic band stretches putting pressure on said rear brace assembly, and said pressure transferring to said user's leg muscles causing them to contract and strengthen after repeated use.

### BRIEF DESCRIPTION OF THE DRAWINGS

The drawings constitute a part of this specification and include exemplary embodiments to the invention, which may be embodied in various forms. It is to be understood that in some instances various aspects of the invention may be shown exaggerated or enlarged to facilitate an understanding of the invention.

FIG. 1 is a perspective view of the invention with rear stretch band in place and the knee in the bent position.

FIG. 2 is a perspective view of the invention with the rear stretch band in place and the knee in the straightened position.

FIG. 3 is a perspective view of the portable rear stretch assembly.

FIG. 4 is side view of the invention showing the rear knee strap.

FIG. 5 is a plan view of the elastic band.

FIG. 6 is a perspective view of the invention with the front stretch band in place.

FIG. 7 is a front perspective view of the invention with the front stretch band in place.

FIG. 8 is a perspective view of the portable front stretch assembly

FIG. 9 is a perspective view of a person attaching the top leg strap.

FIG. 10 is a perspective view of the top leg strap attached.

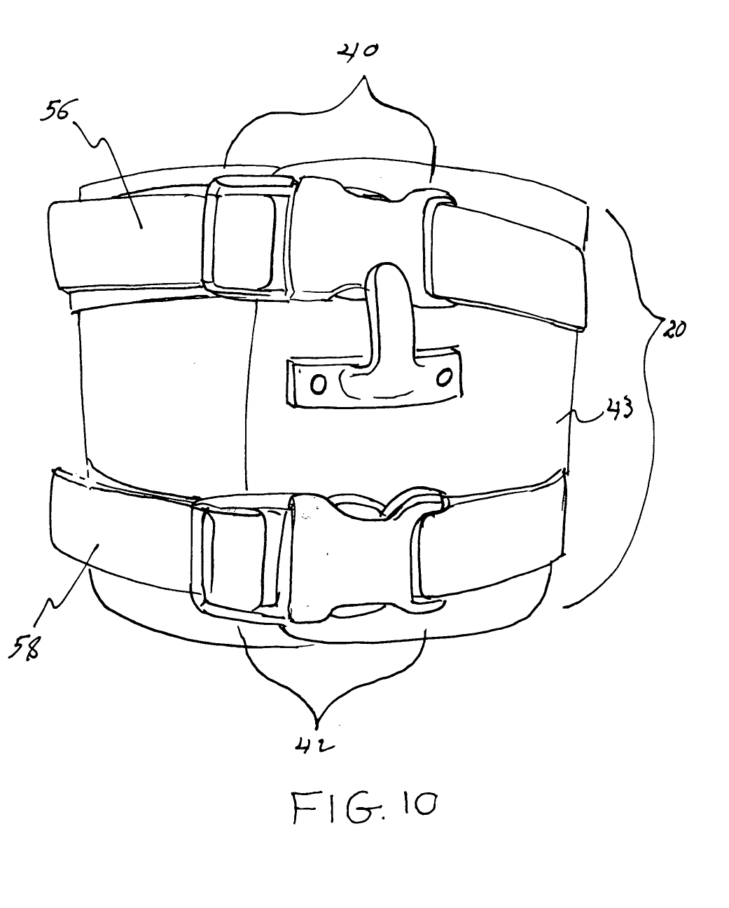

FIG. 11 is a perspective view of the leg strap and brace assembly.

### DETAILED DESCRIPTION OF THE PREFERRED EMBODIMENTS

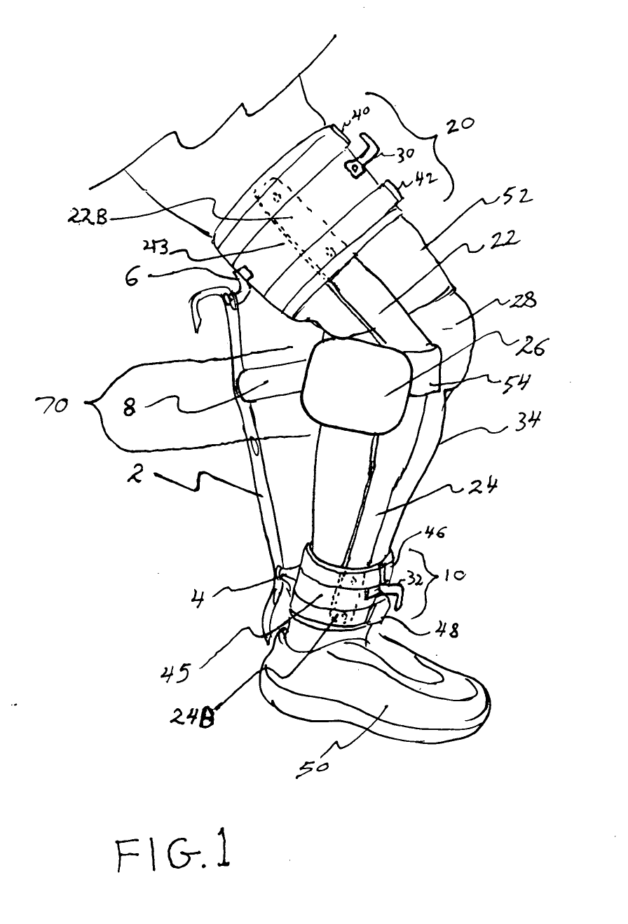

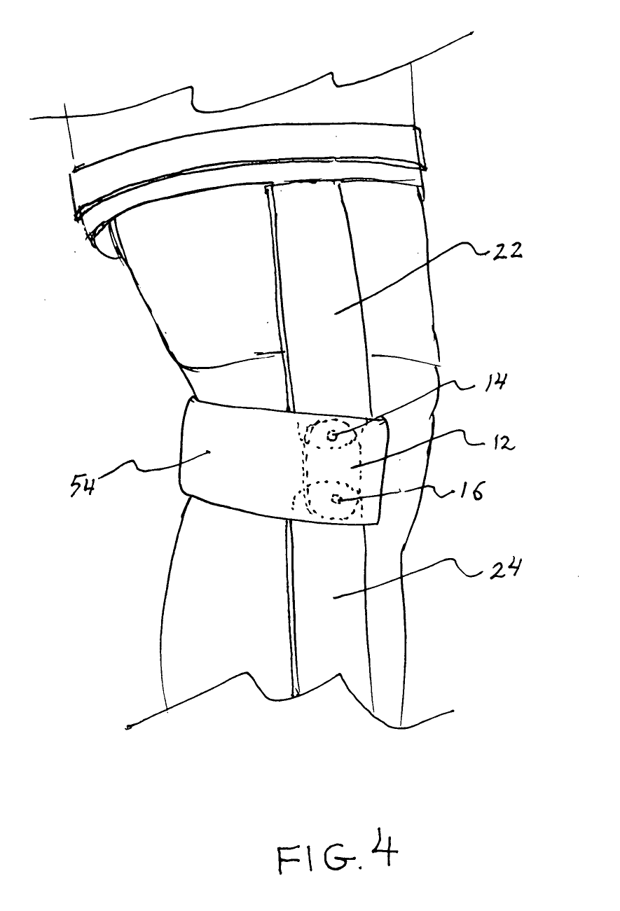

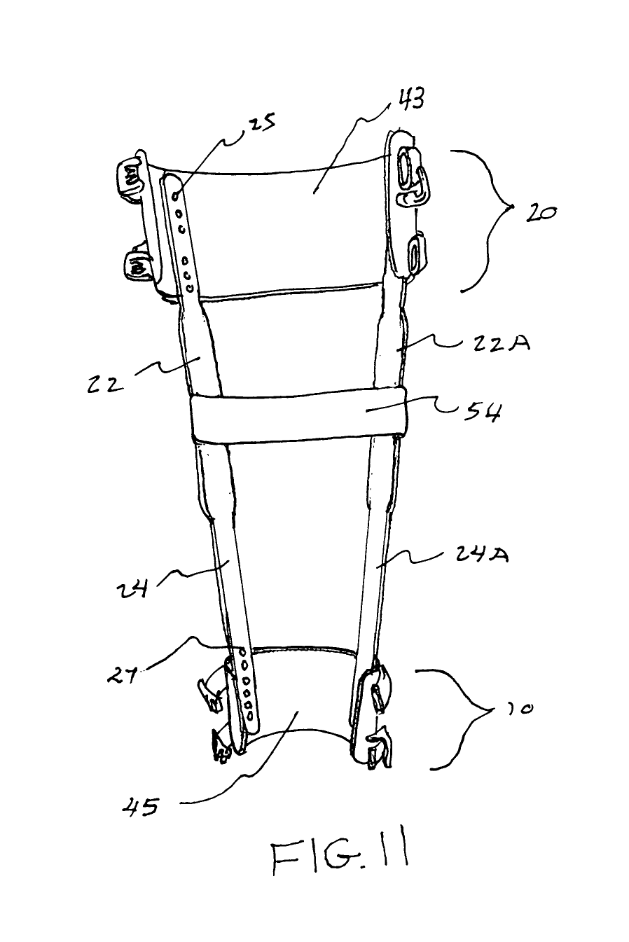

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner. Referring now to FIG. 1 we see a side view of a person's leg 34 and wearing the leg brace of the present invention. A top flexible leg strap assembly 20 is attached to a pair of rigid leg braces. Each leg brace is comprised of an upper half 22 and a lower half 24. Both left and right leg braces can be seen clearly in FIG. 11 and are shown as left brace 22, 24 and right brace 22A and 24A. The rigid leg brace members are pinned as shown by dotted lines 14, 16 in FIG. 4 to an intermediate plate 12 that connects upper brace member 22 to lower brace member 24 via rotatable pin joints 14, 16. FIG. 4 also clearly shows rear knee joint strap 54, which prevents the leg brace members 22, 24 from being pushed forward when under pressure from elastic band 2. Referring back to FIG. 1, leg brace portion 22 extends to the top of nylon leg strap 43 as shown by dotted line 22B and to the bottom of leg strap 45 as shown by dotted line 24B. The leg brace members 22, 24 are fixedly attached to the nylon leg straps 43, 45 so that they prevent the two leg strap assemblies 10, 20 from sliding towards the knee joint when resistive force is applied by strap 2. Nylon strap 43 is approximately five inches wide and nylon strap 45 is approximately four inches wide.

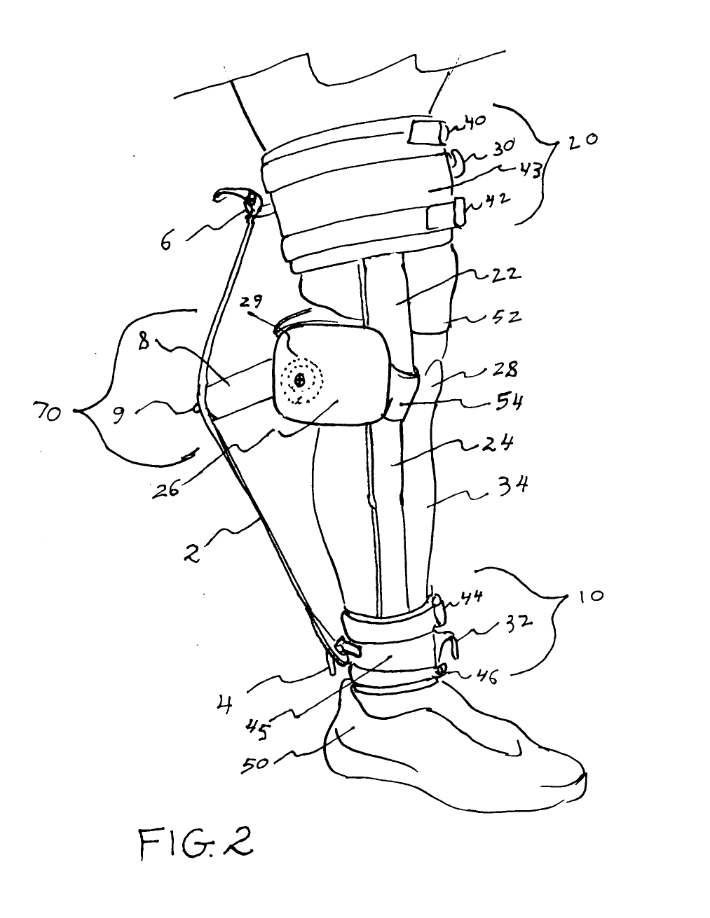

Elastic band 2 is attached to L hook 6 on the upper leg strap 43 and L hook 4 on the lower leg strap 45. Rear leg brace assembly 70 is portable and can be placed between the strap 2 and the rear of the user's knee 28 where rear knee strap 54 is located. The outwardly facing horizontal post 8 of portable brace assembly 70 engages strap 2 so that when the user straightens his or her leg 34, as shown in FIG. 2, the strap 2 is stretched and thereby puts a resistive force on the users muscles associated with the knee joint, specifically the rectus femoris, vastus lateralis, vastus intermedius and vastus medialis muscles. By repeated bending and straightening of the knee joint, the user can help rehabilitate the muscles associated with the knee joint after knee surgery. The present invention can be used as a leg strengthening exercise for non-surgical knee injuries as well. The exercise can be done by an individual without the help of an assistant. The rigid leg braces 22, 24 are preferably made of aluminum and the leg straps 43, 45 are made of plastic sheet nylon type six, approximately one sixteenth of an inch thick, so the entire assembly is light weight. The nylon straps 43, 45 have enough flexibility to be able to adjust to a person's thigh and ankle, but are also rigid enough that they will not deform when resistive pressure is applied by elastic band 2.

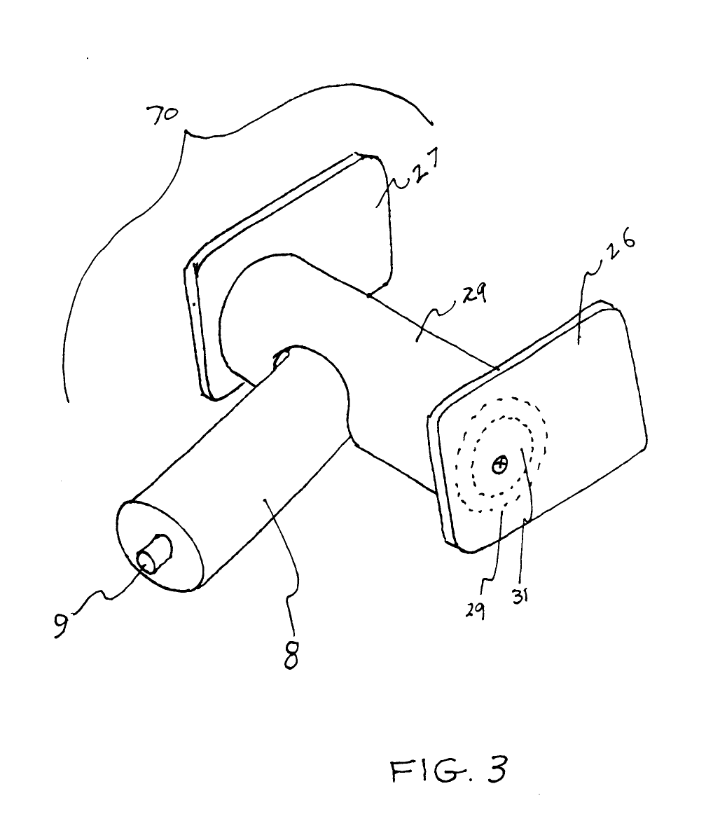

Side plates 26, 27 on portable knee brace assembly 70 help keep the knee from slipping off cross bar 29 as shown in FIG. 3. Crossbar 29 is comprised of a padded layer as shown by dotted line 29 and a solid rod portion as shown by dotted line 31. Extension post 9 engages and protrudes through an aperture 2A in elastic strap 2 thereby holding the post 8 in place.

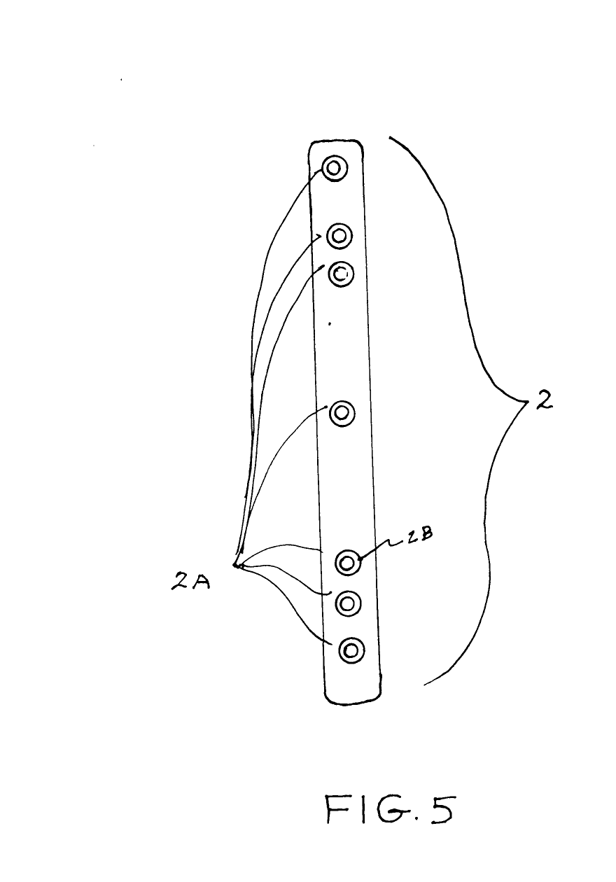

Elastic strap 2 is shown clearly in FIG. 5. A series or apertures 2A are each reinforced by a standard metal or plastic grommet 2B. The strap is made of highly elastic material such as latex rubber. The strap can be made in a variety of thicknesses to provide varying degrees of resistance. Additionally, the user can adjust the length of the strap 2 by choosing which aperture 2A to use to engage L hooks 4, 6. The shorter the distance of the elastic strap, the more resistance is applied to the user's leg muscles. Because the strap can be easily removed and replaced, the user always has the option to increase or decrease resistive force by adjusting the strap length or by replacing one strap with another thinner or thicker one. Another method of applying resistive force, in place of the strap 2, is to incorporate a torsion spring at the joint location of the leg brace. The torsion spring would perform the same effect as the elastic band, however, my experiments have shown that the elastic band is preferable because is provides a smoother range of resistance and can be easily replaced by other more or less resistive straps.

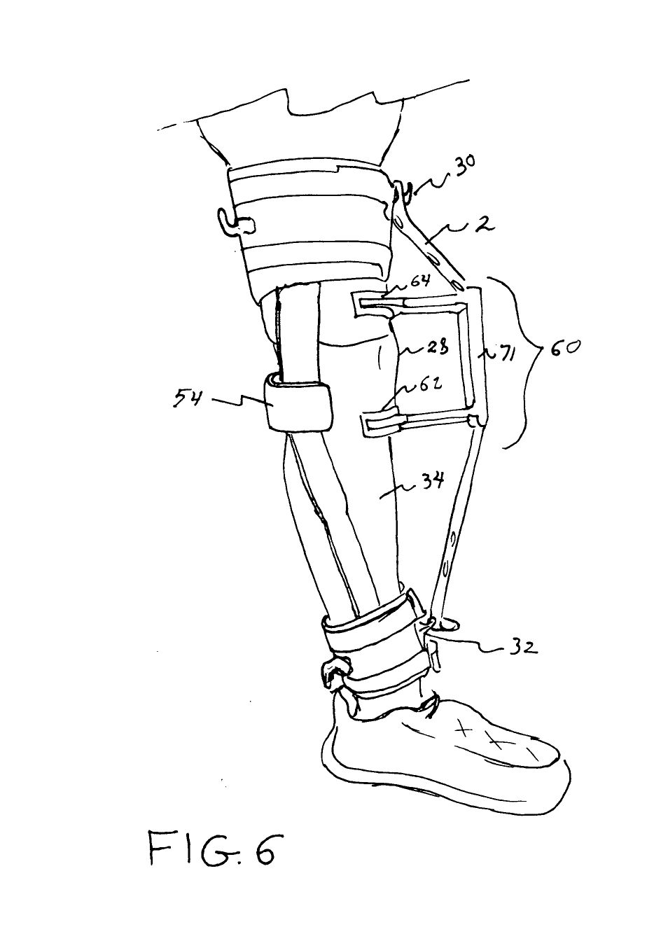

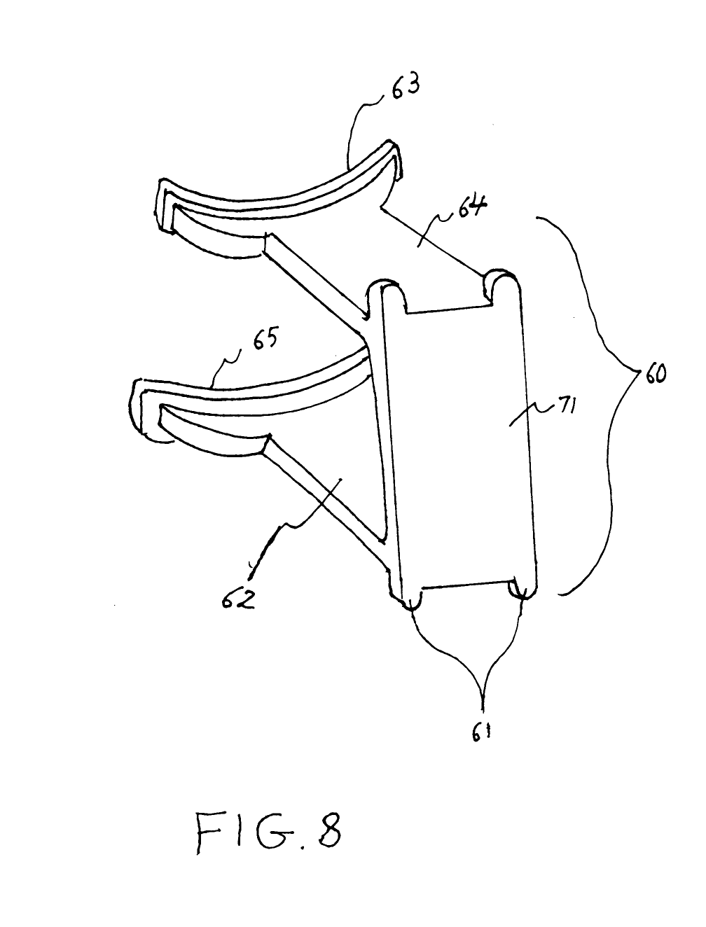

FIG. 6 shows a side view of the invention where the user has attached the elastic straps to the front L hooks 30, 32 and incorporated portable front brace assembly 60 to provide a passive stretching of the knee joint to help straighten a person's leg after knee surgery. It is common for a person to have a difficult time making his or her leg straight after knee surgery. This configuration of the invention helps the muscles and knee joint passively stretch into position. It is an important and novel feature of the present invention that one device can perform both functions of providing resistance to muscles around the knee when used in one mode and providing a stretching of the muscles and passive knee extension when used in the second mode. FIG. 8 shows a clear view of the portable front brace assembly 60. It is comprised of a top and bottom horizontal plate 64, 62 and a vertical plate 71. The vertical plate has tabs 61 at each corner to help retain the elastic strap 2. The spacing of the top plate 64 and the bottom plate 62 is such that the user's knee 28 is spared from any contact, which would be uncomfortable after knee surgery. Padded members 63, 65 interact with the user's leg just above and below the knee and provide comfort when the resistive force is applied by strap 2.

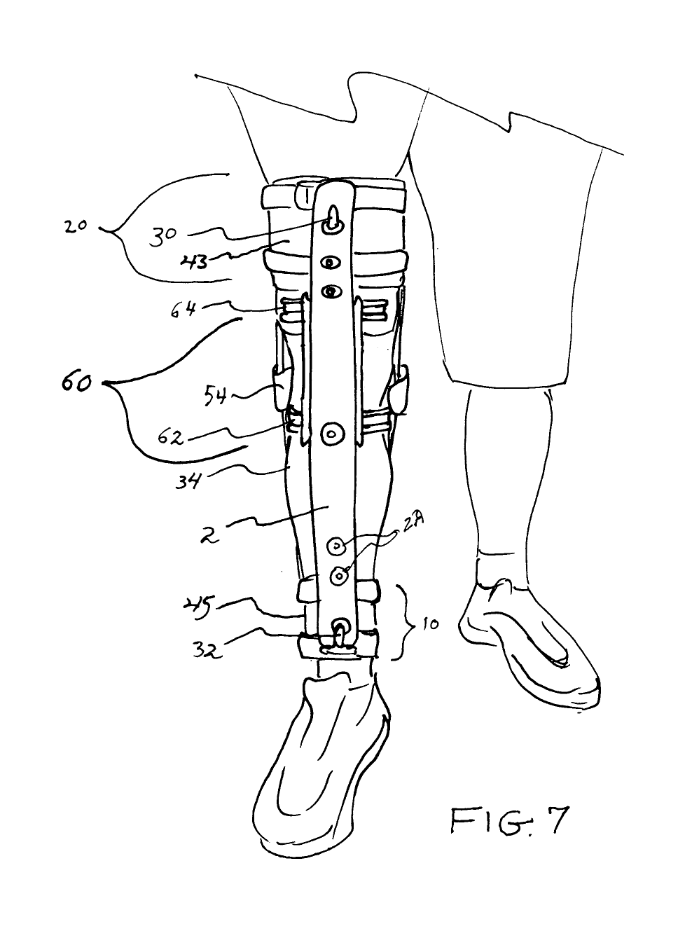

FIG. 7 shows a front view of the front brace assembly in operation. The user can increase the stretching and straightening of the leg by disengaging apertures 2A and engaging apertures that would make the working length of the strap 2 shorter.

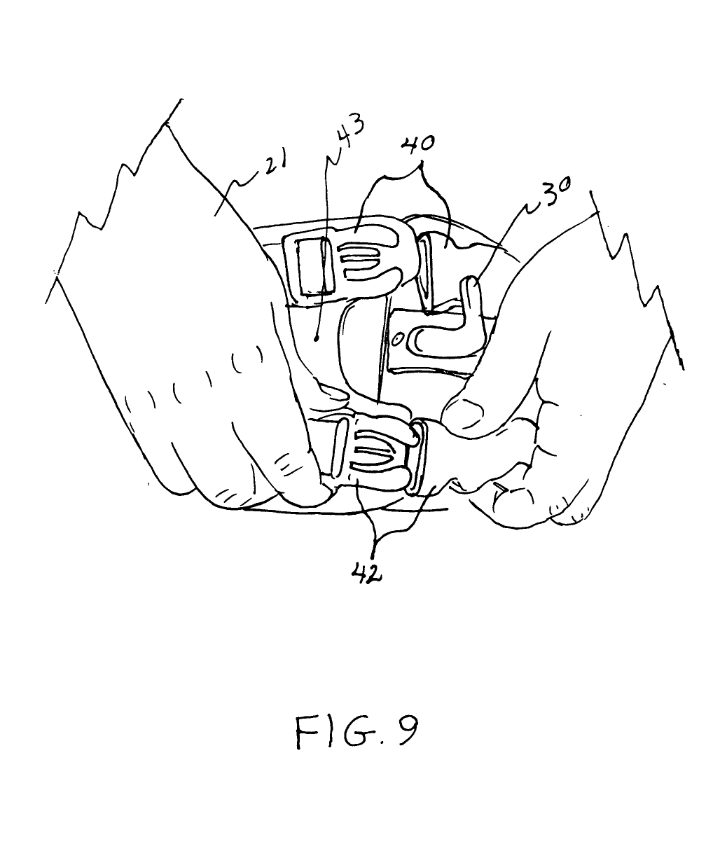

FIG. 9 shows a user 21 connecting one side of the nylon leg strap 43 with the other forming a circular pattern around the user's thigh. The attachment buckles 40, 42 are standard snap fasteners. They are attached to polyester straps 56, 58 which are fixedly attached to the nylon strap 43 as shown in FIG. 11, which shows the snaps 40, 42 in their closed position. The loose ends of polyester straps 56, 58 can be pulled by the user to tighten the nylon leg strap 43 around the user's thigh. The same holds true for lower leg strap assembly 10 where nylon strap 45 is tightened around the user's ankle by closure snaps 46, 48 and tightened in a similar way as upper leg strap assembly 20.

FIG. 11 shows the leg brace assembly in its open, ready to use position. Multiple apertures 25 in the rigid brace 22 allow the user to adjust the height of nylon strap 43, which is held brace 22 by standard fasteners that extend through both the strap 43 and the brace 22. The same holds true for lower leg strap 45 where apertures 27 allow the user to adjust the height of the strap 45 thereby allowing a person to adjust the entire device to the user's leg length.

While the invention has been described in connection with a preferred embodiment, it is not intended to limit the scope of the invention to the particular form set forth, but on the contrary, it is intended to cover such alternatives, modifications, and equivalents as may be included within the spirit and scope of the invention as defined by the appended claims.

What is claimed is:

1. knee rehabilitation exercise device comprising:
a left rigid leg brace;
a right rigid leg brace;
a rear knee strap;
an upper leg strap assembly;
a lower leg strap assembly;
a portable rear brace assembly;
an elastic resistance band;
said left and right leg brace members each comprised of an upper member and a lower member rotatably pinned together at the area of the knee joint;
said left and right leg brace attached at their uppermost ends to said upper leg strap assembly;
said left and right leg brace attached at their lowermost ends to said lower leg strap assembly;
said upper leg strap assembly including a rear facing upwardly turned L hook;
said lower leg strap assembly including a rear facing downwardly turned L hook;
said upper and lower leg strap assemblies able to be formed into a circular shape by a user so that said upper leg strap assembly fits around said user's thigh and said lower leg strap assembly fits around the user's ankle;
said upper and lower leg strap assemblies releasably secured to said user's leg by standard attachment means;
said rear knee strap fixedly held at said pinned knee joint to said left and right led brace;
said portable rear brace assembly held in place behind said user's knee joint by said elastic resistance band when said upper and lower L hooks engage with an upper and a lower aperture in said band;
so that when said user straightens his knee from a bent position, said elastic band stretches putting pressure on said portable rear brace assembly and
said pressure transferring to said user's leg muscles causing them to contract and strengthen after repeated use.

2. Knee rehabilitation exercise device as claimed in claim 1 further comprising a portable front brace assembly;
said upper leg strap assembly including a front facing upwardly turned L hook;
said lower leg strap assembly including a front facing downwardly turned L hook;
said elastic resistance band stretching from said upper L hook, over said front leg brace assembly and terminating at said lower L hook so that a leg straightening pressure is placed on said knee joint

3. Knee rehabilitation exercise device as claimed in claim 1 wherein said portable rear leg brace assembly is comprised of a padded first horizontal bar, a second horizontal bar centrally attached to said first horizontal bar and left and right knee retaining panels fixedly attached to the right and left ends of said first horizontal bar;
said second horizontal bar terminating at its free end in a elastic band aperture receiving post so that said first horizontal bar rests behind said user's knee joint and said second horizontal bar extends outward to interact with said elastic band causing said band to stretch and apply resistive pressure to said user's leg muscles.

4. Knee rehabilitation exercise device as claimed in claim 2 wherein said portable front leg brace assembly is comprised of an upper horizontal brace panel, a lower horizontal brace panel and a vertical brace panel connecting the two
said upper and lower horizontal brace panels terminating at their free ends in a padded arcing portion that conforms with said user's leg so that said upper horizontal panel resides just above said user's knee and said lower horizontal panel resides just below said user's knee;
said vertical panel including extending tabs on all four corners that act as guides for said elastic band when said band is stretched over said vertical panel.

5. Knee rehabilitation exercise device as claimed in claim 1 wherein said upper and lower leg strap assemblies are each comprised of a flexible sheet plastic nylon strap;
said upper strap approximately five inches wide and said lower strap approximately four inches wide;
said nylon straps removably retained by standard polyester belting and standard snap fastening buckles attached to said belting.

6. Knee rehabilitation exercise device as claimed in claim 1 wherein said elastic strap provides resistance and strengthening to the user's rectus femoris, vastus lateralis, vastus intermedius and vastus medialis muscles.

7. Knee rehabilitation exercise device as claimed in claim 2 wherein said elastic strap provides passive stretching the said user's knee joint and surrounding muscles.
